(19) United States
(12) Patent Application Publication
(10) Pub. No.: US 2019/0070557 A1
(43) Pub. Date: Mar. 7, 2019

(54) This biotechnology perpetully grows algae biomass capturing carbon using renewable electricity

(71) Applicant: Nathan Noah Morrison, Winnetka, CA (US)

(72) Inventor: Nathan Noah Morrison, Winnetka, CA (US)

(21) Appl. No.: 15/731,990

(22) Filed: Sep. 5, 2017

(51) Int. Cl. B01D 53/84; B01D 53/62; H01M 10/46; C12M 1/00

(52) U.S. Cl. CPC B01D 53/84; B01D 53/62; H01M 10/465; C12M 23/38; C12M 23/50; H02S 99/00; C12M 43/08; B01D 2251/95; B01D 2258/06; H01M 2220/10; C12M 29/04

(57) ABSTRACT

Atmospheric carbon has risen above 400 parts per million (ppm) crossing a threshold that endangers climate stability. Greenstone Device biotechnology enables consumers, businesses, municipalities and nations to mitigate their carbon output by providing a platform for capturing emitted carbon directly from the atmosphere, and converting that carbon into organic biomass fertilizer.

### BACKGROUND OF THE INVENTION

Problem Solved: Atmospheric carbon has risen above 400 parts per million (ppm) crossing a threshold that endangers climate stability. Greenstone Device biotechnology enables consumers, businesses, municipalities and nations to mitigate their carbon output by providing a platform for capturing emitted carbon directly from the atmosphere, and converting that carbon into organic biomass fertilizer.

### DETAILED DESCRIPTION OF THE INVENTION

This invention recaptures emitted carbon from the atmosphere and converts it into organic biomass fertilizer.

The claimed invention differs from what currently exists. Current legislation and carbon capture policy have focused on reduction efforts primarily. This invention is the first of its kind to provide private and public entities with a viable methodology for carbon capture.

Competitor projects have been ‘one-off’ installation prototypes for high-end green architecture and have not been designed for wide implementation or mass production.

Greenstone Device biotechnology is the opposite of a one-off design prototype, this concept is meant for mass production using modular systems that enable high volume biomass production for the private consumer. The biotechnology includes remote access, enabling maintenance and monitoring to a network of global carbon capture systems from a central hub.

As a byproduct of capturing carbon the Greenstone Device biotechnology produces organic biomass that can be used in the following ways; carbon neutral fertilizer, aquaculture, livestock feed and nutrient supplements.

A brief description of the several views of the attached drawings is below.

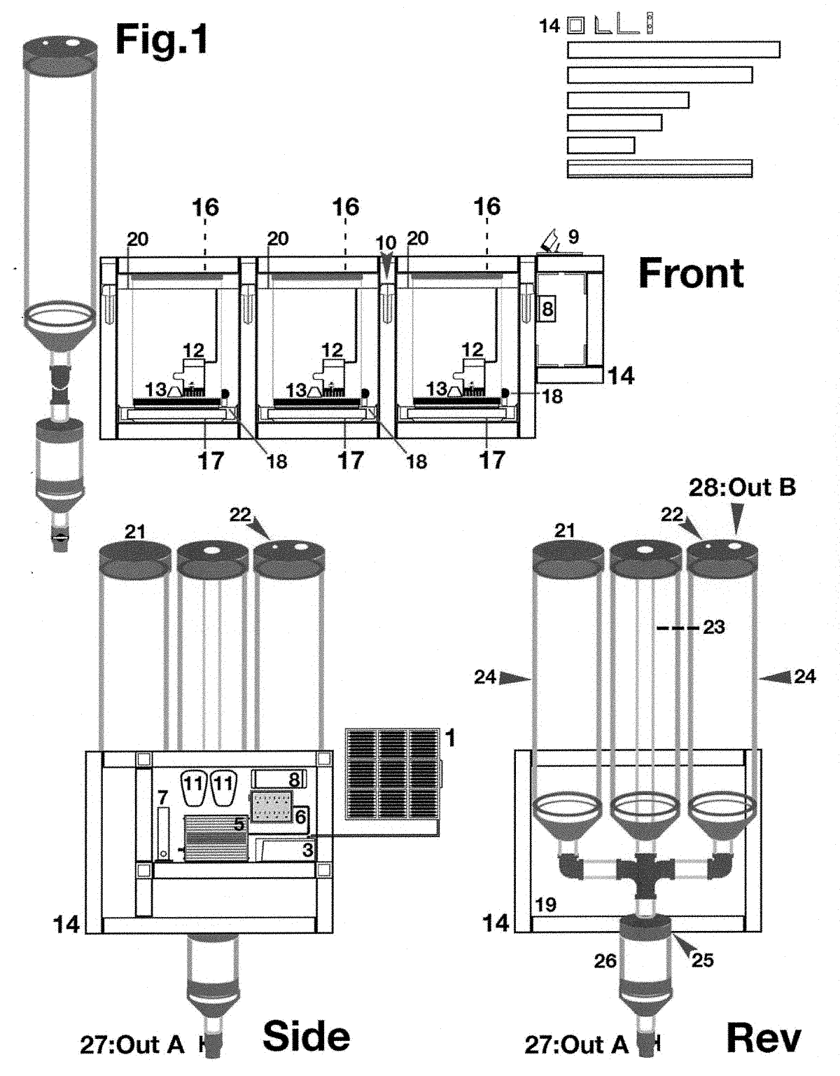

FIG. 1 depicts Greenstone Device biotechnology fully assembled, from a Front, Side, and Reverse perspective.

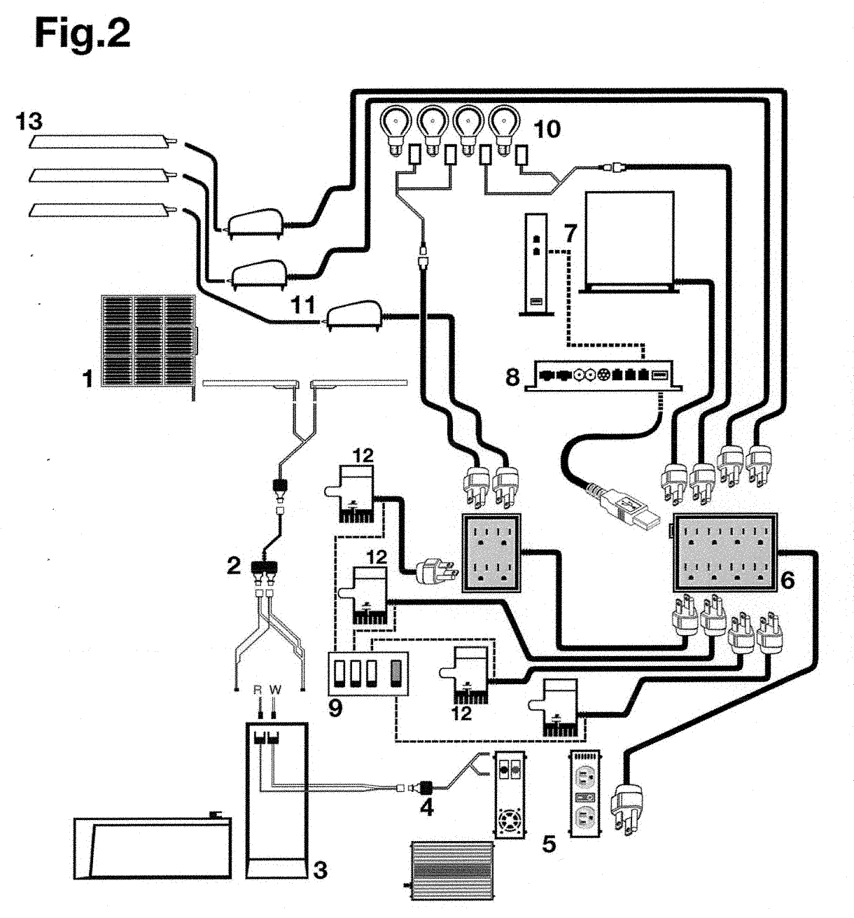

FIG. 2 shows the power throughput, or electrical wiring diagram, illustrating how the components described below connect together.

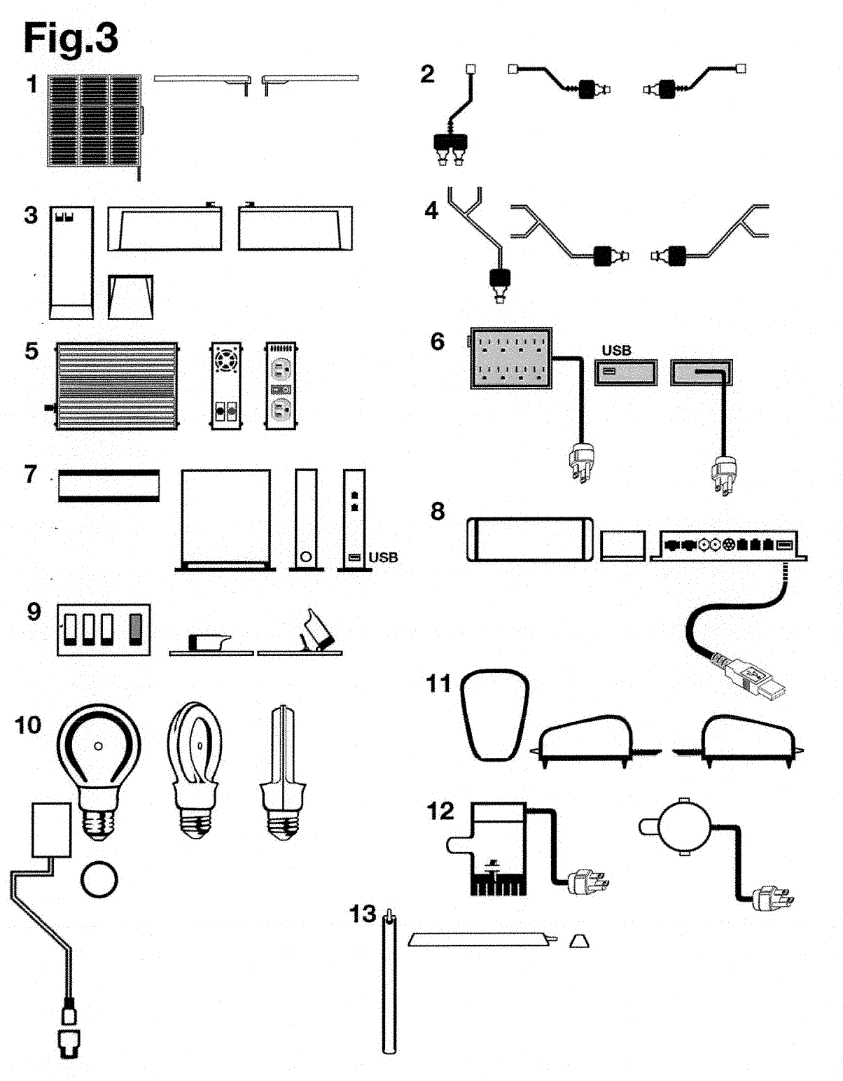

FIG. 3 provides individual component illustrations for Items 1-13 described below.

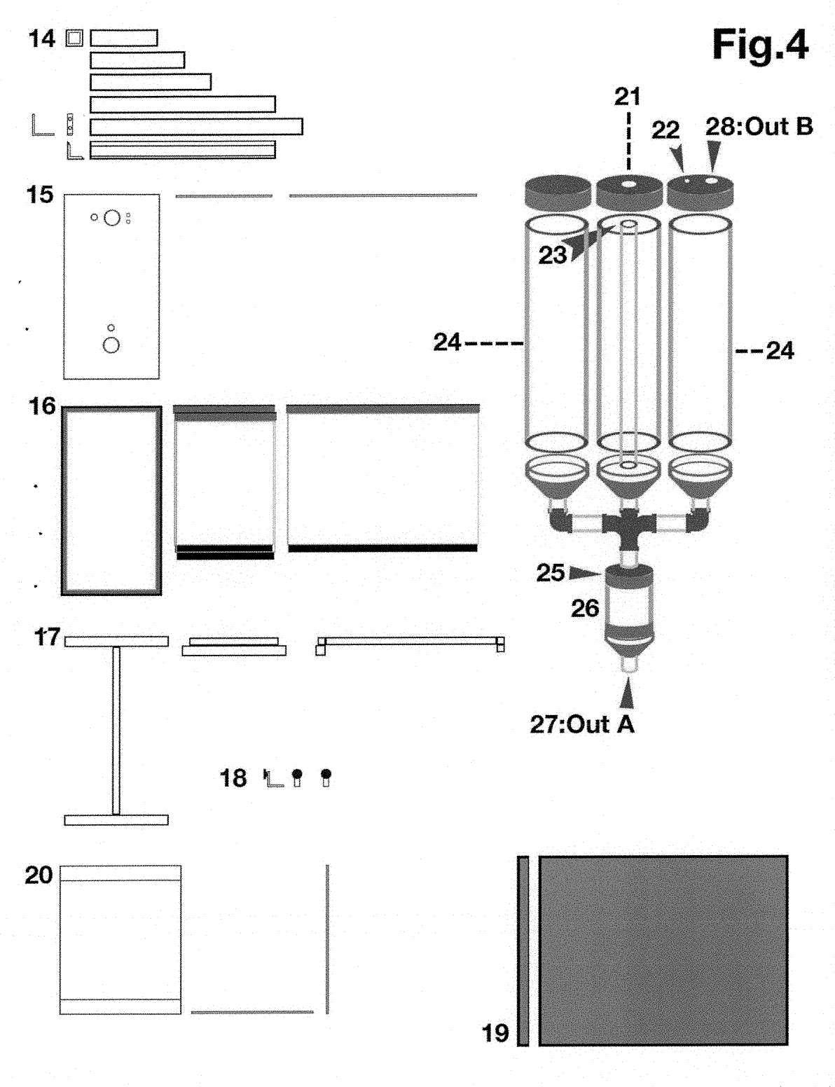

FIG. 4 provides individual component illustrations for Items 14-28 described below.

The individual component Items described on the attached drawings in FIGS. 1-4 include:

1. Renewable Input (Solar, Wind, Etc.)

2. Panel Docking Port

3. DC Battery

4. Battery Docking Port

5. Inverter

6. Outlet Relay

7. WiFi Receiver

8. Aquacontroller

9. Switchplate

10. Lighting Array

11. Air pumps

12. Tank Evacuation Pumps

13. Air Stones

14. BioReactor Housing Structure

15. Permeable Bioreactor Lid

16. Bioreactor Tank

17. Bioreactor Sled

18. Tank Footing Stabilizer

19. Greenstone Device Housing Panels

20. Hermetic Sealing Panel

21. Mass Extraction Separation System

22. Nutrient Feed Reservoir Inlet

23. Biomass Gravity Filter

24. Water Volume Chambers

25. Mass Extraction Removable Cap

26. Mass Volume Chamber

27. OUT A: Biomass Collection

28. OUT B: Water Return

Relationship Between the Components:

The Renewable Input(s) (Solar, Etc.) (1) May be attached physically to the machine, or to an adjacent structure, and then wired to the machine. This component provides the DC Battery (3) with charge. The Panel Docking Port (2) allows for a solar panel array or a single Renewable Input (1) to connect to a single battery terminal. The DC Battery (3) accepts power from Renewable Input (1), stores power and provides power to all electrical systems. Intended to be physically located at BioReactor Housing Structure (14). The Battery Docking Port (4) is an electrical connector for the DC Battery (3) allowing for routine maintenance access point.

The Inverter (5) is physically positioned between the DC Battery (3) and the AquaController (8). The Outlet Relay (6) is power strip that allows for automated control of individual outlets. It is physically positioned adjacent to the AquaController (8). The WiFi Receiver (7) connects the AquaController (8) to a local wireless network to enable remote access and automated control. The AquaController (8) uses the SNT Algae Growth Algorithm to govern growth and internal systems and to enable remote access and automated control. The Switchplate (9) attached to the BioReactor Housing Structure (14) enables On-Site access and manual control for emergency and routine maintenance. The Lighting Array (10) is isolated from the Bioreactor Tank (16), providing light to the growth environment. The lighting array works in complement with reflective surfaces adhered to the internal surface of the Bioreactor Housing Structure (14), maximizing radiance and minimizing photon leakage. The Air Pumps (11) is intended to pump air (atmosphere) into the Bioreactor Tank (16). The pump is mounted within the Bioreactor Housing Structure (14). Tank Evacuation Pumps (12) deliver biomass yield to the Mass Extraction Separation System (21). These automated plumbing components are physically mounted within the Bioreactor Tank (16). The Bioreactor Sled (17) is the structural mount for the Bioreactor Tank (16) allowing for manual access to each individual growth vessel. The Tank Footing Stabilizer (18) is a horizontal catch for the Bioreactor Sled (17) reducing slide movement. The Greenstone Device Housing Panels (19) are the modular outer wall coverings attached to the Bioreactor Housing Structure (14) that reduce temperature fluctuation and protect the growth environment from invasive species. The Hermetic Sealing Panel (20) is translucent barrier that allows for signage opportunities and hermetically seals the growth environment against foreign contamination. Each individual panel is attached to the Bioreactor Housing Structure (14) and located at the face of each module. The Mass Extraction Separation System (21) accepts biomass yield and separates water from wet algae biomass in the Mass Processor (26). This system allows for on-site biomass harvest and water reclamation.

The Nutrient Feed Reservoir Inlet (22) is attached to the OUT B: Water Return (28) and provides access for feed delivery. The Biomass Gravity Filter (23) optimizes and channels water flow to aid the separation process happening internally in the Mass Extraction Separation System (21). The Water Volume Chambers (24) accepts excess water from the Mass Extraction separation System (21) and isolates the water from the biomass. The Biomass Extraction Removable Cap (25) allows for manual access and removal of the Mass Volume Chamber (26) or the algae biomass harvest. The Mass Volume Chamber (26) accepts filtered biomass from the Biomass Gravity Filter (23) and the Water Volume Chamber (24). The OUT A: Biomass Collection (27) is a manual valve that allows for on-site algae biomass harvest. This pipe connection point is located at the base of the Mass Volume Chamber (26). The OUT B: Water Return (28) this pump network returns reclaimed water to the first stage growth volume.

How the Invention Works:

Sunlight is gathered through the photovoltaic solar panels in the solar array and translated into usable renewable energy for the electrical components of the Greenstone Device. The Bioreactor Tanks house a monoculture of algae for perpetual growth, drain and carbon capture. These modular volumes drain collectively into the Mass Extraction Separation System for on-site algae biomass collection, local application or sale. Data collected through the control system reports to a central SNT network that will establish compliance with the required authorities on the client's behalf.

How To Make the Invention:

Various Solar Panels, Electric Batteries, Air Pumps and Water Pumps exist on the market, and the Greenstone Device could potentially utilize these existing subsystems. The subsystems described in this machine are custom made in our workshop. To create the machine, these required components are assembled by a certified craftsman according to the drawings attached to this patent.

All elements and methodologies depicted and described are required for the machine to function as designed. No components are optional for proper operation.

How to Use the Invention:

Algae biomass produced at the OUT A: Biomass Collection point would be available for landscape crews, local agriculture or international partnerships to meet carbon reduction targets.

Additional uses for organic biomass include but are not limited to: carbon neutral fertilizer, aquaculture, livestock feed and nutrient supplements.

1. An apparatus for sequestering carbon dioxide within algae biomass, comprising: a renewable electrical input, a panel docking port, DC battery, battery docking port, inverter, outlet relay, WiFi receiver, aquacontroller, switchplate, lighting array, air pumps, tank evacuation pumps, air stones, bioreactor housing structure, permeable bioreactor lid, bioreactor tank, bioreactor sled, tank footing stabilizer, Greenstone Device housing panels, hermetic sealing panel, mass extraction separation system, nutrient feed reservoir inlet, biomass gravity filter, water volume chambers, mass extraction removable cap, mass volume chamber, OUT A: biomass collection, OUT B: water return; wherein said renewable electrical input is wired to the panel docking port, which in turn is wired to the DC battery allowing the renewable electrical input to charge the DC battery; and wherein the DC battery is docked within the battery docking port, and is wired to the inverter, which translates DC to AC electricity and provides power to the outlet relay; and wherein the outlet relay accepts power cables from and provides power to: the Wifi receiver, aquacontroller, lighting array, air pumps, tank evacuation pumps, and to the mass extraction separation system; and wherein the Wifi receiver, aquacontroller, lighting array, air pumps, tank evacuation pumps and mass extraction separation system are governed by the aquacontroller and are connected to the switchplate for optional manual governance; and wherein said permeable bioreactor lid seals the bioreactor tank which is docked on the bioreactor sled and secured by the tank footing stabilizer and surrounded by the bioreator housing structure, which is enclosed by Greenstone Device housing panels and hermetic sealing panel; and wherein the bioreactor tank contains air stones which are fed air by the air pumps, and the bioreactor tank is connected to the mass extraction separation system, and wherein the mass extraction separation system contains a mass extraction removable cap, and a nutrient feed reservoir inlet connected to water volume chambers connected to a biomass gravity filter which processes biomass into the mass volume chamber to be extracted via the OUT A biomass collection, and which processes water via the OUT B water return back into the bioreactor tank.
